(19) United States
(12) Patent Application Publication
(10) Pub. No.: US 2014/0332436 A1
(43) Pub. Date: Nov. 13, 2014

(54) DISPOSABLE WEARING ARTICLE AND PACKAGED ASSEMBLY THEREOF

(71) Applicant: UNICHARM CORPORATION, Ehime (JP)

(72) Inventors: Kenichi Sasayama, Kagawa (JP); Makoto Ichikawa, Kagawa (JP); Kunihiko Katsuragawa, Kagawa (JP); Kazuo Ukegawa, Kagawa (JP)

(21) Appl. No.: 14/125,486

(22) PCT Filed: Jun. 28, 2012
(86) PCT No.: PCT/JP2012/004210

§ 371 Date: Dec. 11, 2013

(30) Foreign Application Priority Data
Jun. 30, 2011 (JP) 2011-146655
Nov. 30, 2011 (JP) 2011-261124

(51) Int. Cl. A61F 13/49; B65D 85/18; A61F 13/551

(52) U.S. Cl. CPC A61F 13/49001; A61F 13/49012; A61F 13/49017; A61F 13/55115; B65D 85/18
USPC 206/494; 604/385.201; 604/385.16; 604/385.3; 604/385.25

(57) ABSTRACT

A disposable wearing article adapted to restrict a possibility that the article might become locally bulky and a packaged assembly of such disposable wearing articles.

A diaper (1) has front and rear waist panels (20, 30) defining front and rear waist regions (2, 3), respectively, and an intermediate panel (40) defining parts of the front and rear waist regions and a crotch region (4). The diaper (1) is folded on itself substantially in a flat condition so that inner sheets (24, 34) of the front and rear waist panels (20, 30) may face each other, respectively, and an inner sheet of the intermediate panel (40) may face itself. Specifically, the diaper (1) is folded on itself along a first fold line (81) at least partially corresponding to an imaginary transverse center line (Q-Q). In the diaper (1) having been folded on itself along the first fold line (81), a crotch midsection (83) in the crotch region (3) has a thickness dimension smaller than a thickness dimension of regions (85) lying in the front and rear waist panels (20, 30).

### TECHNICAL FIELD

The present disclosure relates to disposable wearing articles and packaged assemblies thereof and, more particularly, to disposable wearing articles such as pants-type disposable diapers, disposable toilet-training pants, disposable incontinent pants or disposable sanitary napkins and packaged assemblies thereof.

### BACKGROUND

Pants-type disposable diapers formed with a waist-opening and a pair of leg-openings and packaged assemblies of these diapers are known. For example, JP H11-113956 A (PTL 1) discloses a pants-type diaper and a method of packaging these diapers including the steps of folding opposite side edges of the diaper onto the side of the front waist region or onto the side of the rear waist region along the fold lines extending in the longitudinal direction and then folding the crotch region along the fold line extending in the transverse direction to package the diapers in the packing bag. JP 2000-42029 A (PTL 2) discloses a packaged assembly of pants-type diapers wherein each of the diapers is folded along the fold line extending across the crotch region in the transverse direction before the two or more diapers are stacked on each other.

### CITATION LIST

### Patent Literature

PTL 1: JP H11-113956 A
PTL 2: JP 2000-42029 A

### SUMMARY OF INVENTION

### Technical Problem

In the above discussed diapers, the absorbent member is located in the crotch region. The absorbent member is usually formed of fluff wood pulp and additional material, and the inventor(s) have recognized that the crotch region might become more bulky than the other regions in which the absorbent member is not located.

### Solution to Problem

This disclosure includes at least first and second aspects.

The first aspect relates to a disposable wearing article having a longitudinal direction and a transverse direction, and including a chassis and a bodily fluid absorbent member. The chassis comprises a skin-facing side, a non-skin-facing side opposite to the skin-facing side, front and rear waist regions, a crotch region extending between these front and rear waist regions, a waist-opening, a pair of leg-openings, an imaginary longitudinal center line bisecting a dimension of the chassis in the transverse direction and an imaginary transverse center line bisecting a dimension of the chassis in the longitudinal direction. The bodily fluid absorbent member lies at least in the crotch region. A first fold line corresponds to or is in a vicinity of the imaginary transverse center line, wherein the wearing article is folded along the first fold line so that the skin-facing side of the front waist region faces the skin-facing side of the rear waist region and the skin-facing side of the crotch region faces itself.

A crotch midsection included within the crotch region and lying on the imaginary transverse center line has a thickness dimension smaller than a thickness dimension of absorbent-free regions which are defined in the front and rear waist regions and in which the bodily fluid absorbent member is not present.

The second aspect relates to a packaged assembly comprising a package sheet and a plurality of disposable wearing articles stacked one on another in the thickness direction of the respective disposable wearing articles, and packaged with the package sheet.

Each of these disposable wearing articles having a longitudinal direction and a transverse direction includes a chassis and a bodily fluid absorbent member. The chassis comprises a skin-facing side, a non-skin-facing side opposite to the skin-facing side, front and rear waist regions, a crotch region extending between these front and rear waist regions, a waist-opening, a pair of leg-openings, an imaginary longitudinal center line bisecting a dimension of the chassis in the transverse direction and an imaginary transverse center line bisecting a dimension of the chassis in the longitudinal direction. The bodily fluid absorbent member lies at least in the crotch region. A first fold line corresponds to or is in a vicinity of the imaginary transverse center line.

A crotch midsection included within the crotch region and lying on the imaginary transverse center line has a thickness dimension smaller than a thickness dimension of absorbent-free regions which are defined in the front and rear waist regions and in which the bodily fluid absorbent member is not present.

Each of the disposable wearing articles is folded along the first fold line so that the skin-facing side of the front waist region may face the skin-facing side of the rear waist region and the skin-facing side of the crotch region may face itself.

### BRIEF DESCRIPTION OF DRAWINGS

FIG. 1 is a perspective view of a disposable diaper as one example of a disposable wearing article in accordance with some embodiments.

FIG. 2 is a developed view of the diaper.

FIG. 3 is an exploded perspective view of the diaper.

FIG. 4 is a front view of the diaper.

FIG. 5 is a scale-enlarged sectional view taken along line V-V in FIG. 4.

FIG. 6 is a scale-enlarged sectional view taken along line VI-VI in FIG. 4.

FIG. 7 is a front view of the diaper in a folded state.

FIG. 8 is an exploded perspective diagram illustrating the diapers stacked on each other.

FIG. 9 is a perspective view of a packaged assembly of the diapers.

FIGS. 10(a)-(b) are diagrams of the diaper in one exemplary folded state.

FIGS. 11(a)-(b) are diagrams of the diaper in another exemplary folded state.

### DESCRIPTION OF EMBODIMENTS

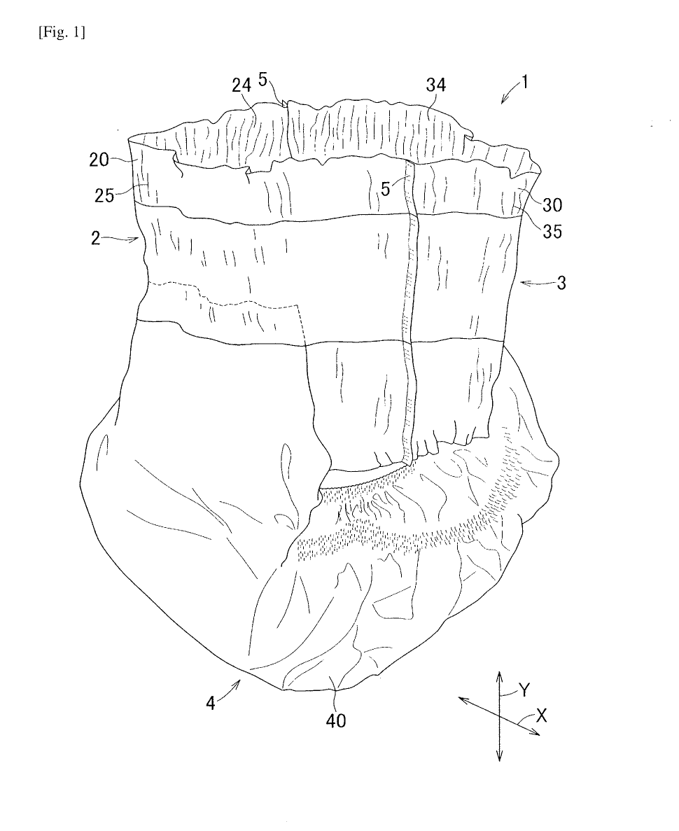

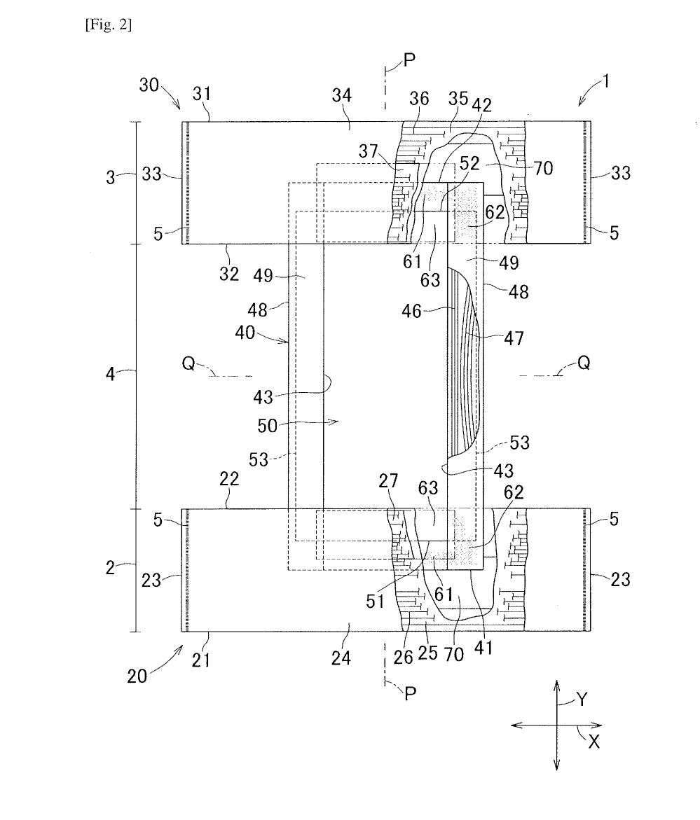

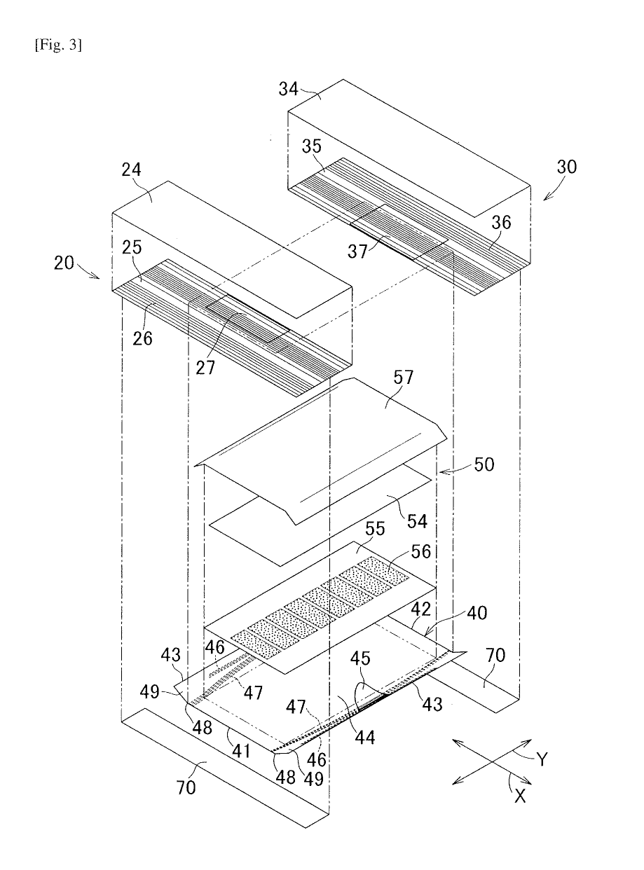

FIG. 1 is a perspective view of a disposable diaper 1 as one example of a disposable wearing article according to some embodiments of this invention, FIG. 2 is a developed plan view of the diaper 1 as viewed from the wearer's body and FIG. 3 is an exploded perspective view of the diaper 1. In FIGS. 2 and 3, respective elastic members are illustrated as they are under tension against contractile force thereof and partially cutaway for convenience of illustration.

Referring to FIGS. 1 through 3, the diaper 1 has a longitudinal direction Y and a transverse direction X being orthogonal to the longitudinal direction Y, and includes a skin-facing side, a non-skin-facing side (i.e., the side facing the wearer's garment), a front waist region 2, a rear waist region 3 and a crotch region 4 extending between the front and rear waist regions 2, 3, wherein these regions 2, 3, 4, are continuous in the longitudinal direction Y. The diaper 1 includes front and rear waist panels 20, 30 defining the front and rear waist regions 2, 3, respectively, and an intermediate panel 40 defining the crotch region 4 and parts of the front and rear waist regions 2, 3, and wherein these front and rear waist panels 20, 30 and the intermediate panel 40 are joined together to define a chassis.

Referring to FIGS. 2 and 3, the front waist panel 20 has an outer end 21 and an inner end 22 both extending in the transverse direction X and opposite side edges 23 extending in the longitudinal direction Y. In a similar way, the rear waist panel 30 has an outer end 31 and an inner end 32 both extending in the transverse direction X and opposite side edges 33 extending in the longitudinal direction Y. The intermediate panel 40 has front and rear ends 41, 42 both extending in the transverse direction X and opposite side edges 43 extending in the longitudinal direction Y. The front and rear ends 41, 42 of the intermediate panel 40 are respectively attached to the front and rear waist panels 20, 30 on the non-skin-facing sides thereof. The opposite side edges 23 of the front waist panel 20 and the opposite side edges 33 of the rear waist panel 30 are joined together to define seams 5. Upon formation of the seams 5, the front and rear waist panels 20, 30 are maintained in an annular configuration, the outer ends 21, 31 cooperate with each other to define a waist-opening, and the inner ends 22, 32 cooperate with the intermediate panel opposite side edges 43 to define a pair of leg-openings, as illustrated in FIG. 1.

The diaper 1 has an imaginary longitudinal center line P-P bisecting a dimension of the diaper 1 in the transverse direction X and an imaginary transverse center line Q-Q bisecting a dimension of the diaper 1 in the longitudinal direction P-P and the diaper 1 is shaped nearly symmetrically about these imaginary longitudinal center line P-P and imaginary transverse center line Q-Q. More specifically, the imaginary transverse center line Q-Q is a bisector for the dimension in the longitudinal direction Y between the outer end 21 of the front waist panel 20 and the outer end 31 of the rear waist panel 30. Similarly, the imaginary longitudinal center line P-P is a bisector for the dimension in the transverse direction X between the opposite side edges 23, 33 of the front and rear waist panels 20, 30.

The front and rear waist panels 20, 30 respectively include inner sheets 24, 34 lying on the skin-facing sides of these panels, outer sheets 25, 35 lying on the non-skin-facing sides of these panels and a plurality of waist elastics 26, 36 disposed between these sheets. The inner sheets 24, 34 and the outer sheets 25, 35 are formed of one of liquid-impermeable or poorly liquid permeable spunbonded/meltblown/spunbonded (SMS) fibrous nonwoven fabrics, spun bonded fibrous nonwoven fabrics, plastic sheets and/or laminates of such nonwoven fabrics and plastic sheets, each having a mass per unit area in a range of about 10 to about 30 g/m².

The waist elastics 26, 36 are attached to be spaced apart from each other in the longitudinal direction Y under tension but to be contractible in the transverse direction X and thereby elasticize the front and rear waist panels 20, 30 in the transverse direction X. As the waist elastics 26, 36, one or more of thread-, strand- or string-like members having rubber elasticity may be used. In this embodiment, elastic threads having a fineness in a range of about 780 to about 940 dtex are used. One of the inner and outer sheets 24, 25 and one of the inner and outer sheets 34, 35 are respectively coated with hot melt adhesives (not shown) with which these inner and outer sheets 24, 25 and 34, 35 are respectively bonded together and at the same time the waist elastics 26, 36 are bonded to these inner and outer sheets.

The waist elastics 26, 36 are attached under tension at a stretch ratio in a range of about 2.0 to about 3.0 to the inner and outer sheets 24, 25 and to the inner and outer sheets 34, 35. The term “stretch ratio” used herein means a ratio of a stretched length to the natural length of the elastic members before these members are stretched and specifically means that the elastic members are attached in the state having been stretched at a ratio in a range of about 2.0 to about 3.0 times of the natural length of these members. While the stretch ratio of two or more waist elastics 26, 36 may be partially varied to adjust the contractile force of these waist elastics 26, 36, a difference between the maximum stretch ratio and the minimum stretch ratio is in a range of about 0 to about 10%. In this way, it is possible to prevent apparent dimensions of the front and rear waist panels 20, 30 in the transverse direction X from being locally and remarkably reduced due to a local increase of the contractile force and also possible to maintain the front and rear waist panels 20, 30 as a whole in substantially rectangular shapes defined by the opposite side edges 23 and the opposite side edges 33, respectively. If the stretch ratio, and therefore contractile force, is higher in a specific elastic member than in the remaining elastic members, the thickness dimension of the portion of the front or rear waist panel 20, 30 corresponding to the specific elastic member will be correspondingly increased. From this viewpoint, preferably, the stretch ratios of the respective elastic members are substantially equalized to equalize the thickness dimension of the front and rear waist panels 20, 30.

While the front and rear waist panels 20, 30 may be elasticized merely by attaching the waist elastics 26, 36 thereto, it is possible to elasticize the front and rear waist panels 20, 30 by use of, for example, an elastic fibrous nonwoven fabric as the front and rear waist panels 20, 30 and it is also possible to combine such an elastic fibrous nonwoven fabric with the waist elastics 26, 36. The elastic fibrous nonwoven fabric may be selected from the group of various types of fibrous nonwoven fabrics such as spun bonded fibrous nonwoven fabrics, melt blown fibrous nonwoven fabrics, heat roll fibrous nonwoven fabrics, spunbonded/meltblown/spunbonded (SMS) fibrous nonwoven fabrics, air-through fibrous nonwoven fabrics, spun laced fibrous nonwoven fabrics and air-laid fibrous nonwoven fabrics. One of them may be separately used or two or more of them may be used in combination. As the constituent resins of these elastic fibrous nonwoven fabrics, for example, an elastomer resin such as polystyrene and polyurethane or a thermoplastic resin such as polyethylene, polypropylene, polyester or acryl may be used. Furthermore, various types of conjugate fibers comprising these resins such as polyethylene/polypropylene, polyethylene/polyethylene terephthalate or polypropylene/polyethylene terephthalate conjugate fibers of core-in-sheath or side-by-side may be also used separately or in combination.

Waist leakage-barrier sheets 27, 37 are disposed between the inner and outer sheets 24, 25 and between the inner and outer sheets 34, 35, respectively. As the waist leakage-barrier sheets 27, 37, liquid-impermeable but moisture-permeable plastic films may be used. The waist leakage-barrier sheet 27 is attached to the front waist panel 20 along the inner end 22 thereof so as to lie in a middle portion thereof as viewed in the transverse direction X. In a similar way, the waist leakage-barrier sheet 37 is attached to the rear waist panel 30 along the inner end 32 thereof so as to lie in a middle portion thereof as viewed in the transverse direction X. The waist leakage-barrier sheets 27, 37 respectively have areas smaller than those of the inner and outer sheets 24, 25; 34, 35. In consequence, both the front and rear waist panels 20, 30 include regions in which the waist leakage-barrier sheets 27, 37 are not present, respectively.

Referring to FIG. 3, the intermediate panel 40 includes an inner sheet 44 lying on the skin-facing side, an outer sheet 45 lying on the non-skin-facing side and first and second leg elastics 46, 47 disposed between these inner and outer sheets 44, 45. As the inner sheet 44, liquid-impermeable but moisture-permeable plastic films may be used and, as the outer sheet 45, poorly-liquid-permeable fibrous nonwoven fabrics may be used.

The intermediate panel 40 has a pair of fold lines 48 extending in the longitudinal direction Y and folded regions 49 to be folded inward in the transverse direction X along the respective fold lines 48 are defined between the opposite side edges 43 and the fold lines 48. In the respective folded regions 49, the inner sheet 44 faces each other. In the respective folded regions 49, the inner sheet 44 is bonded to itself with adhesives (not shown) in vicinities of the front and rear ends 41, 42 of the intermediate panel 40.

As the first and second leg elastics 46, 47, one or more thread-, strand- or string-like members having rubber elasticity may be used. These first and second leg elastics 46, 47 are respectively attached under tension but in a contractible manner in the longitudinal direction Y to the intermediate panel 40 so that the intermediate panel 40 may be elasticized in vicinities of the opposite side edges 43 thereof, specifically, in the respective folded regions 49 in the longitudinal direction Y. The first leg elastics 46 are attached to the intermediate panel 40 along the opposite side edges 43 and the second leg elastics 47 are attached thereto rather along the respective fold lines 48. The first and second leg elastics 46, 47 are bonded to at least one of the inner and outer sheets 44, 45 with hot melt adhesive or the like (not shown).

The first and second leg elastics 46, 47 attached to the intermediate panel 40 along one of the opposite side edges 43 are not continuous or connected with the leg elastics 46, 47 extending along the other of the opposite side edges 43. In other words, none of elastic members extend between the opposite side edges 43 of the intermediate panel 40 in the transverse direction X. In consequence, the intermediate panel 40 should not contract in the transverse direction X and, in this way, it is possible to prevent the inner and outer sheets 44, 45 from contracting in the transverse direction X particularly in the middle portion in the transverse direction X along the imaginary transverse center line Q-Q.

The front and rear ends 41, 42 of the aforementioned intermediate panel 40 are bonded to the respective non-skin-facing sides of the front and rear waist panels 20, 30 by bonding means such as hot melt adhesives. More specifically, as illustrated in FIG. 2, first bonding regions 61 extending in the transverse direction X along the front and rear ends 41, 42, respectively, and second bonding regions 62 on both sides of the respective first bonding regions 61 as viewed in the transverse direction X so as to extend in the longitudinal direction Y. Between the first and second bonding regions 61, 62, non-bonding regions 63 not coated with adhesive or the like are defined. The non-bonding regions 63 are defined substantially in the middle zones, respectively, as viewed in the transverse direction X. Each set of the first and second bonding regions 61, 62 and the non-bonding regions 63 defines a U-shape opening toward the crotch region 4. The intermediate panel 40 is joined to the front and rear waist panels 20, 30 so that the opposite side edges of the respective waist leakage-barrier sheets 27, 37 attached to the front and rear waist panels 20, 30 may be in coincidence with the opposite side edges of the non-bonding regions 63.

In the diaper 1, the intermediate panel 40 is joined to the front and rear waist panels 20, 30 on the non-skin-facing sides thereof and, in consequence, bodily fluids such as urine should not move to the skin-facing sides of the front and rear waist panels 20, 30 and the wearer's skin should not suffer from rashes due to direct contact with such bodily fluids even when bodily fluids move along the intermediate panel 40 in the longitudinal direction Y toward the front and rear waist panels 20, 30. The intermediate panel 40 is folded along the fold lines 48, the folded regions 49 defined in this manner are bonded to the front and rear waist panels 20, 30 and the first and second leg elastics 46, 47 are attached to these folded regions 49 so that the vicinities of the opposite side edges 43 may be put in close contact with the wearer's body.

The intermediate panel 40 is joined to the waist panels 20, 30 with the first bonding region 61 and the second bonding regions 62 so that pockets opening toward the crotch region 4 are defined by the non-bonding regions 63, respectively. These pockets are adapted to retain body waste moving in the longitudinal direction Y along the intermediate panel 40 and thereby to prevent body waste from leaking beyond the waist-opening. The waist leakage-barrier sheets 27, 37 attached to the respective middle portions of the front and rear waist panels 20, 30 are adapted to prevent body waste from permeating through the pockets and leaking toward the non-skin-facing side (garment-facing side).

A bodily fluid absorbent member 50 is attached to the skin-facing side of the intermediate panel 40. The bodily fluid absorbent member 50 has front and rear ends 51, 52 extending in the transverse direction X and opposite side edges 53 extending in the longitudinal direction Y wherein the front and rear ends 51, 52 lie inside the front and rear ends 41, 42 of the intermediate panel 40 in the longitudinal direction Y and the opposite side edges 53 lie inside the respective fold lines 48 in the transverse direction X. The folded regions 49 of the intermediate panel 40 may be put in close contact with the wearer's body to keep the bodily fluid absorbent member 50 spaced apart from the wearer's body. Consequently, it is possible to protect the wearer against rashes due to direct contact of the wearer's skin with body waste discharged onto the bodily fluid absorbent member 50 and, at the same time, to prevent body waste from leaking out beyond the leg-openings' peripheral edges.

Referring again to FIG. 3, the bodily fluid absorbent member 50 includes inner and outer sheets 54, 55, an absorbent core material 56 disposed between these inner and outer sheets 54, 55 and a skin-facing side liner 57 lying on the skin-facing side so as to cover the inner and outer sheets 54, 55 and the core material 56. The inner and outer sheets 54, 55 may be formed of, for example, a liquid-permeable spunbonded/meltblown/spunbonded (SMS) fibrous nonwoven fabric. As the core material 56, insoluble superabsorbent polymer particles may be used.

Between the inner and outer sheets 54, 55, interruption zones are formed to restrict migration of the core material. In this embodiment, the interruption zones are formed of hot melt adhesives. More specifically, the outer sheet 55 is coated over its entire area with hot melt adhesives (not shown) and the core material is dispersed thereon except on the front and rear ends and the opposite side edges so as to define the interruption zones along the front and rear ends and the opposite side edges. In addition, one or more of such interruption zones in which none or substantially none of the core material is dispersed are provided so as to extend in the transverse direction and spaced apart from each other in the longitudinal direction Y. In the interruption zones in which none of the core material is dispersed, the outer sheet 55 is directly bonded to the inner sheet 54 to define a plurality of dispersed core material containing sub-regions. In this way, the core material should not spill out from the sub-region or sub-regions even if the core material 56 comes unstuck from the hot melt adhesive. While the core material is bonded to at least one of the inner and outer sheets 54, 55 with hot melt adhesives in this embodiment, such fixation of the core material is not essential for this invention. Alternatively, the inner and outer sheets 54, 55 may be partially bonded to each other so as to define several spaces adapted to retain the core material therein, respectively. In this case, at least one portion of the core material is not secured and movable within the respective spaces but the movement of the core material out of the respective spaces is prevented.

While superabsorbent polymer particles are used as the core material 56 in this embodiment, it is possible to use fluff wood pulp or a mixture of fluff wood pulp and superabsorbent polymer particles as the core material 56. When using the core material in the form of the mixture, a content rate of superabsorbent polymer particles is preferably adjusted to be as higher as possible in order that the thickness dimension of the bodily fluid absorbent member 50 may be reduced and thereby the thickness dimension of the crotch region 4 may be reduced, as will be described later in detail.

Cover sheets 70 adapted to cover the front and rear ends 41, 42 are attached to the non-skin-facing side of the intermediate panel 40 having been joined to the front and rear waist panels 20, 30. As the cover sheets 70, for example, spun bonded/melt blown/spun bonded fibrous nonwoven fabrics or spun bonded fibrous nonwoven fabrics may be used. The cover sheets 70 attached in this manner function to prevent the hands or legs or garment of the wearer from being caught by the front and rear ends 41, 42 and, in consequence, to prevent the intermediate panel 40 from being peeled off from the front and rear waist panels 20, 30.

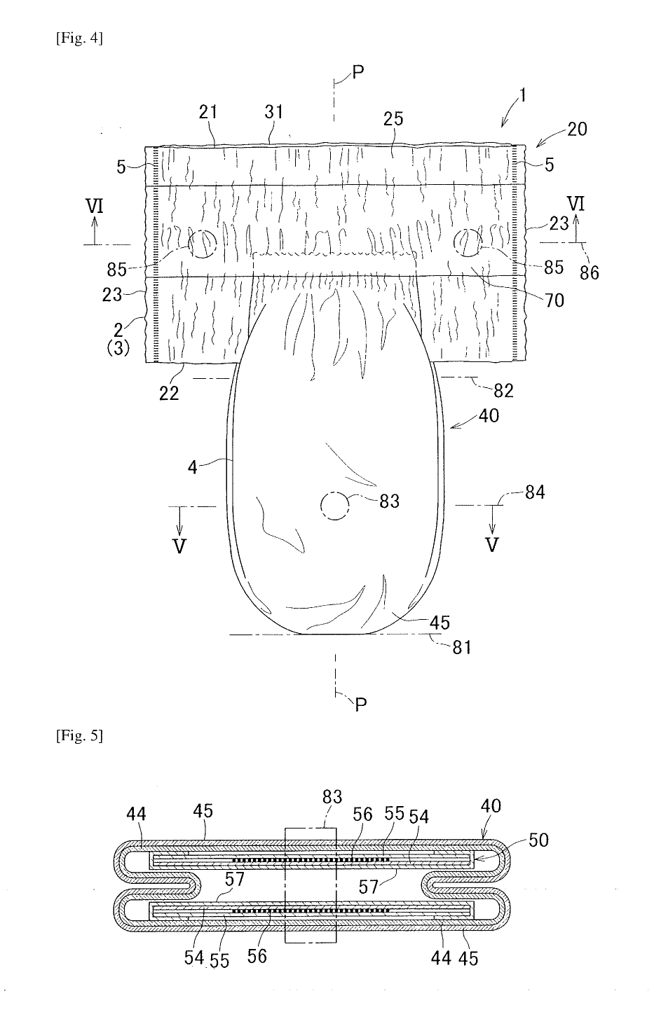

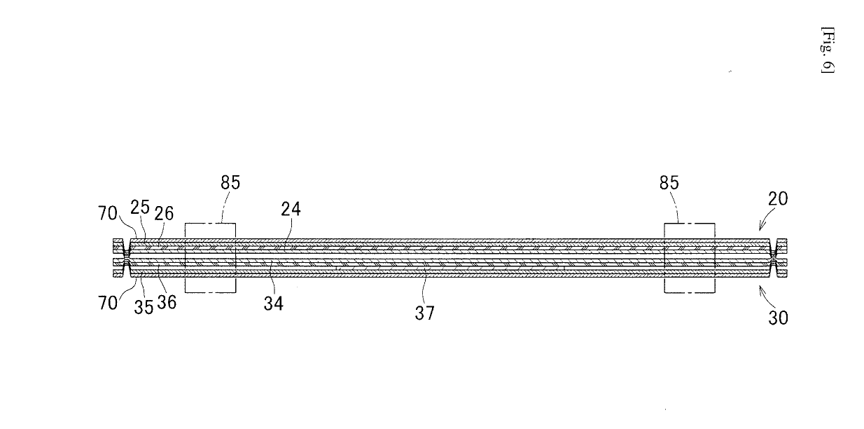

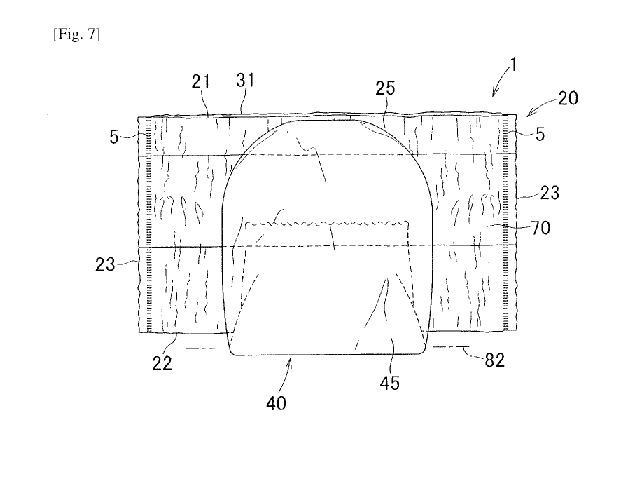

FIGS. 4 through 7 illustrate the folded diaper 1 before the diaper 1 is actually used. FIG. 4 is a front view of the diaper 1 folded along a first fold line 81, FIG. 5 is a scale-enlarged sectional view taken along line V-V in FIG. 4, FIG. 6 is a scale-enlarged sectional view taken along line VI-VI in FIG. 4 and FIG. 7 is a front view of the diaper folded along a second fold line 82.

As illustrated in FIGS. 4 through 6, the diaper 1 having the front and rear waist panels 20, 30 joined together along the seams 5 is put in a substantially flat state in which the respective inner sheets 24, 34 (i.e., the skin-facing side) of the front and rear waist panels 20, 30 face each other and the inner sheet 44 (i.e., the skin-facing side) of the intermediate panel 40 faces itself. Up to this time point, the diaper 1 has already been at least partially folded along a first fold line 81 corresponding to the imaginary transverse center line Q-Q. In other words, the diaper 1 has been folded so that the dimension of the diaper 1 in the longitudinal direction Y may be substantially bisected.

In the diaper 1 folded along the first fold line 81, a thickness dimension of a crotch midsection 83 is smaller than a thickness dimension of respective absorbent-free regions 85 defined in the front and rear waist panels 20, 30. More specifically, the thickness dimension of the crotch midsection 83 is in a range of about 2.0 to about 8.0 mm, preferably in a range of about 2.0 to about 5.0 mm and the thickness dimension of the respective absorbent-free regions 85 is in a range of about 4.0 to about 12.0 mm, preferably in a range of about 5.0 to about 10.0 mm. The crotch midsection 83 is a region defined around an intersecting point of the imaginary longitudinal center line P-P and an imaginary line 84 which is closest to the first fold line 81 among imaginary lines quartering a dimension in the longitudinal direction Y from the outer ends 21, 31 to the first fold line 81. The absorbent-free regions 85 are the regions defined on an imaginary line 86 bisecting a dimension of the first and second waist panels 20, 30 in the longitudinal direction Y and not overlapping the bodily fluid absorbent member 50, i.e., the regions in which the bodily fluid absorbent member 50 is not present. In this embodiment, the thickness dimension was measured in a pair of the absorbent-free regions 85 respectively lying on the opposite outer sides of the intermediate panel 40 in the transverse direction X and an average value was calculated. In this regard, it is also possible to measure the thickness dimension in the absorbent-free regions 85 lying on the imaginary line 86 defined as a line bisecting a length dimension in the longitudinal direction Y of the respective seams 5 or to measure the thickness dimension in the other regions not overlapping the bodily fluid absorbent member 50.

Referring to FIG. 5, the crotch midsection 83 is a region defined by a pair of layered structures each comprising the inner and outer sheets 44, 45 of the intermediate panel 40 and the bodily fluid absorbent member 50, but including none of the folded regions 49, the first leg elastics 46 and the second leg elastics 47.

Referring to FIG. 6, the absorbent-free regions 85 are regions respectively defined by a pair of layered structures one of which includes the inner sheet 24, the outer sheet 25, the waist elastics 26 and the cover sheet 70 and the other includes the inner sheet 34, the outer sheet 35, the waist elastics 36 and the cover sheet 70. The thickness dimension was measured with the waist elastics 26, 36 not under tension but left contract.

<Measuring Method for Thickness>

The thickness dimensions of the crotch midsection 83 and the absorbent-free regions 85 was measured with use of PEACOCK accurate measuring instruments (Thickness Gauge of large size type J-B manufactured by OZAKI MFG. CO., LTD.). The measurement was carried out with the use of a disc-probe having a diameter of 50 mm under a measurement pressure of about 2N. Three measurements were made and averaged.

In the diaper 1 in the aforementioned state, a cantilever bending resistance of the crotch region 4 inclusive of the crotch midsection 83 was in a range of about 15 to about 140 mm.

<Measuring Method for Cantilever Bending Resistance>

In accordance with JIS L1096—Cantilever method, test pieces (having a dimension of 50 mm in the transverse direction X and a dimension of 150 mm in the longitudinal direction Y) were cut out from the intermediate panel 40 inclusive of the crotch mid-sections 83 of the diapers 1 and measurements were conducted on the skin-facing surface and the non-skin-facing surface of the respective test pieces. Three measurements were made for each of the skin-facing side and the non-skin-facing side, and the measured values were averaged.

Referring again to FIG. 4, the diaper 1 in the aforementioned state is further folded along a second fold line 82 extending across the crotch region 4 in the transverse direction X. The second fold line 82 corresponds to or is in a vicinity of a line bisecting a dimension in the longitudinal direction Y between the outer ends 21, 31 and the first fold line 81. In this embodiment, a length dimension in the longitudinal direction Y between the inner ends 22, 32 and the first fold line 81 is larger than a length dimension in the longitudinal direction Y of the front and rear waist panels 20, 30. In consequence, the second fold line 82 lies in the crotch region 4, i.e., on the intermediate panel 40. The second fold line 82 is not necessarily a pre-formed line; it is a fold line formed when the diaper 1 having been folded along the first fold line 81 as illustrated in FIG. 4 is further folded in two. It is not necessary to put this second fold line 82 in coincidence with the aforementioned bisector. In view of this, it is not essential that the second fold line 82 coincides with the aforementioned bisector so long as the second fold line 82 lies in the crotch region 4.

Referring to FIG. 7, upon folding the diaper 1 along the second fold line 82, the outer sheet 25 (i.e., the non-skin-facing side) of the front waist panel 20 and the outer sheet 45 (i.e., the non-skin-facing side) of the intermediate panel 40 face each other. While the intermediate panel 40 is folded onto the front waist panel 20 in this embodiment, it is possible to fold the intermediate panel 40 onto the rear waist panel 30. In this case, the outer sheet 45 (i.e., the non-skin-facing side) of the intermediate panel 40 will face the outer sheet 35 (i.e., the non-skin-facing side) of the rear waist panel 40. After the diaper 1 has been folded along the second fold line 82, the outer shape of the diaper 1 is substantially contoured by the outer ends 21, 31, the second fold line 82 and the opposite side edges 23.

In the diaper 1 in the aforementioned state, the crotch midsection 83 is dimensioned so as to be sufficiently thin to restrict a possibility that a total thickness dimension might become significantly bulky even after the intermediate panel 40 has been folded along the first fold line 81 and the second fold line 82. Particularly because the crotch midsection 83 is dimensioned to be thinner than the front and rear waist panels 20, 30, it is possible to prevent the regions of the front and rear waist panels 20, 30 on which the intermediate panel 40 is folded from becoming significantly thick due to the presence of the intermediate panel 40. In addition, the crotch midsection 83 includes none of the elastic members particularly extending in the transverse direction X and, in consequence, the crotch midsection 83 should not become bulky due to wrinkles appearing when the elastic members contract. By primarily using superabsorbent polymer particles as the core material of the bodily fluid absorbent member and using no other material, such as fluff wood pulp or, if used, limiting the content of the other material to minimum percentage, the dimension of the crotch midsection 83 in the thickness direction can be further reduced. The thickness dimension may be reduced in this manner to alleviate bothersome handling of the used diaper 1 for disposal thereof.

Cantilever bending resistance of the crotch region 4 may be set to a range of about 15 to about 140 mm to avoid a problem that the crotch region 4 might be easily wrinkled or rucked. With such crotch region 4 free from wrinkles and rucks, the diaper 1 can be smoothly unfolded to put the diaper 1 on the wearer's body. In addition, a possibility that the wearer's skin might be irritated by wrinkles of the diaper 1 is alleviated.

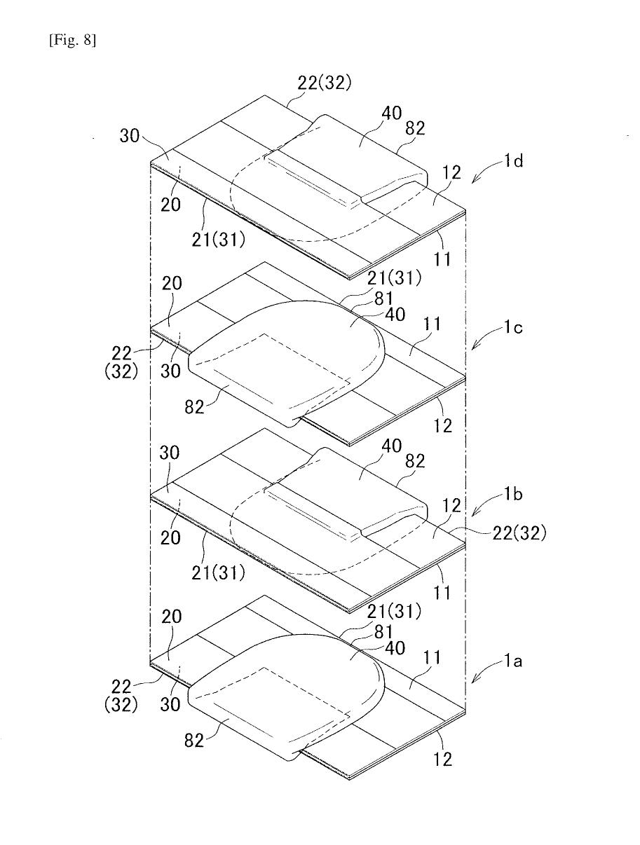

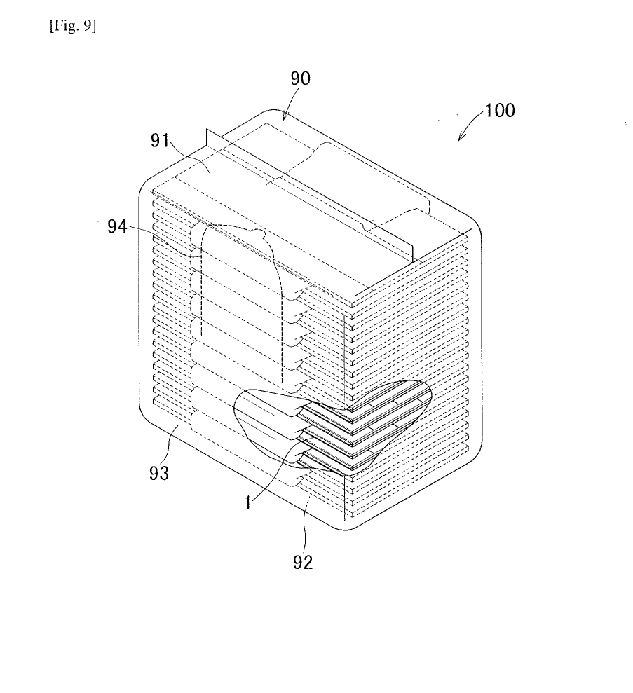

FIG. 8 is an exploded perspective diagram illustrating a plurality of the diapers stacked on each other. As illustrated, each of the diapers 1a, 1b, 1c and 1d having been folded along the first and second fold lines 81, 82 respectively, has opposite first and second surfaces 11, 12. The folded intermediate panel 40 is exposed more on the first surface 11 than on the second surface 12. Specifically, an exposed area of the intermediate panel 40 is larger on the first surface 11 than on the second surface 12 and an exposed area of the front waist panel 20 or the rear waist panel 30 is larger on the second surface 12 than on the first surface 11. In this embodiment, the intermediate panel 40 is folded so as to overlap the front waist panel 20 and, in consequence, the rear waist panel 30 is exposed on the second surface 12.

The adjacent diapers 1a, 1b are stacked one on another so that the first surfaces 11 of the respective diapers 1a, 1b face each other and the adjacent diapers 1c, 1d are also stacked one on another so that the first surfaces 11 of the respective diapers 1c, 1d. The outer ends 21, 31 of the diaper 1a face the second fold line 82 of the diaper 1b, and the outer ends 21, 31 of the diaper 1b face the second fold line 82 of the diaper 1c. In this manner, respective postures of the adjacent diapers in the stack are inverted in the longitudinal direction Y. By stacking a plurality of the diapers in this manner, a dimension of the stack in the thickness direction can be substantially equalized in the longitudinal direction Y as well as in the transverse direction X of the diapers and the stack can be stabilized against a possibility of collapsing. A packaged assembly may be maintained substantially in a cubic or box shape and a plurality of such packaged assemblies stacked one on another can be stabilized against a possibility of collapsing. Such packaged assemblies allow a space for storage to be reduced and thereby to improve the efficiency of commodity distribution and storage.

FIG. 9 is a perspective view, partially cutaway for convenience of illustration, of a packaged assembly 100 of the diapers with use of a package sheet. As illustrated, the diapers 1 stacked one on another in the manner as has been described above are packaged with a flexible package sheet 90 to form the packaged assembly 100.

The package sheet 90 has an upper surface 91, a bottom surface 92 and side surfaces 93 extending between these upper and bottom surfaces 91, 92 and a plurality of the diapers 1 are stacked on one another between the bottom surface 92 and the upper surface 91 in so-called flat pile fashion. In such packaged assembly 100, the diapers 1 are compressed in the direction of stacking and packed with the package sheet 90 to be maintained in the compressed state.

In the packaged assembly 100 as has been described above, each of the folded diapers 1 has a substantially rectangular outer shape and a substantially uniform thickness in the transverse direction X as well as in the longitudinal direction Y of the diaper 1, i.e., without a remarkable difference in the thickness. In consequence, these diapers may be stacked one on another to obtain a substantially cubic or box-shaped assembly. Also after packaged with use of the package sheet 90, such cubic assembly can be maintained and even when a plurality of such packaged assemblies 100 are stacked one on another, the stacked packaged assemblies are stabilized without an anxiety of easily collapsing.

The package sheet 90 is formed with an indicator and/or tearable line 94, such as a perforated line, along which the package sheet 90 can be torn. The indicator line 94 may be formed at least on the upper surface 93 and, in the embodiment illustrated in FIG. 9, the indicator line 94 is on the upper surface 91 and the side surface 93. More specifically, the indicator line 94 extends from the side surface 93 upward to the upper surface 91 and then extends downward to the side surface 93 so as to describe a U-shape. When it is desired to pull out one of the diapers 1 from the package sheet 90, the package sheet 90 may be torn along the indicator line 94 from the upper surface 91 toward the side surface 93. An opening is formed in the upper surface 91 and/or side surface 93 and therefore the individual diapers 1 stacked one on another can be pulled out one by one for actual use thereof. The diapers 1 are in a state compressed in a vertical direction as viewed in FIG. 9 and therefore it might be difficult to pull out the diaper 1 from the bottom of the stack but it will be easy to pull out the diaper 1 from the opened upper surface 91 and/or side surface 93 one by one since the compressing force is reduced in vicinities of the partially opened upper surface 91 and/or side surface 93.

The indicator line 94 may be formed at least on the upper surface 91 so that the diapers 1 can be pulled out one by one from the side of the upper surface 91. In this regard, it is also possible to form the indicator lines at a plurality of locations.

While the diapers 1 each having been folded along the first fold line 81 and the second fold line 82 (See FIG. 7) are stacked one on another and packaged according to this embodiment, it is also possible to stack and to package the diapers 1 in the state as illustrated in FIG. 4, i.e., in the state in which the respective diapers 1 have been folded along the first fold line 81 but not along the second fold line 82. In this case, a thickness difference between the crotch midsection 83 of the crotch region 4 and the absorbent-free regions 85 of the front and rear waist regions 2, 3 in the individual diapers 1 is preferably small. More preferably, this thickness difference is about 4.0 mm or less.

Thickness dimensions of the crotch midsection 83 and the absorbent-free regions 85 were measured in the same manner as the aforementioned thickness measurement. Thickness of the crotch midsection 83 and the absorbent-free regions 85 was dimensioned to be sufficiently small to prevent particularly the front and rear waist regions 2, 3 from becoming remarkably bulky when a plurality of the diapers 1 are stacked one on another. Consequently, it is possible not only to make the packaged assembly compact but also to maintain the packaged assembly as a whole in a rectangular parallelepiped shape.

As the package sheet 90, flexible synthetic resin may be used. Specifically, polyethylene film having a mass per unit area in a range of about 20 to 70 g/m² may be used. When superabsorbent polymer particles are used as the core material 56 of the absorbent member 50, a mass per unit area of the resinous material constituting the package sheet 90 used for the packaged assembly may be reduced in comparison with the case in which, for example, fluff wood pulp is used as the core material 56.

On the assumption that fluff wood pulp is used as the core material and compressed to be packaged with use of the package sheet 90, a restoring force of fluff wood pulp will thrust the package sheet 90 from the inside. In order that the package sheet 90 may resist against to such thrusting force without being broken, the mass per unit area of the resinous material is increased and thereby to enhance the strength thereof. In contrast, superabsorbent polymer particles are not easily compressed and consequently free from causing a remarkable thrusting force from the inside. Thus, the package sheet 90 should not be broken even when the mass per unit area of the resinous material for the package sheet 90 is relatively small.

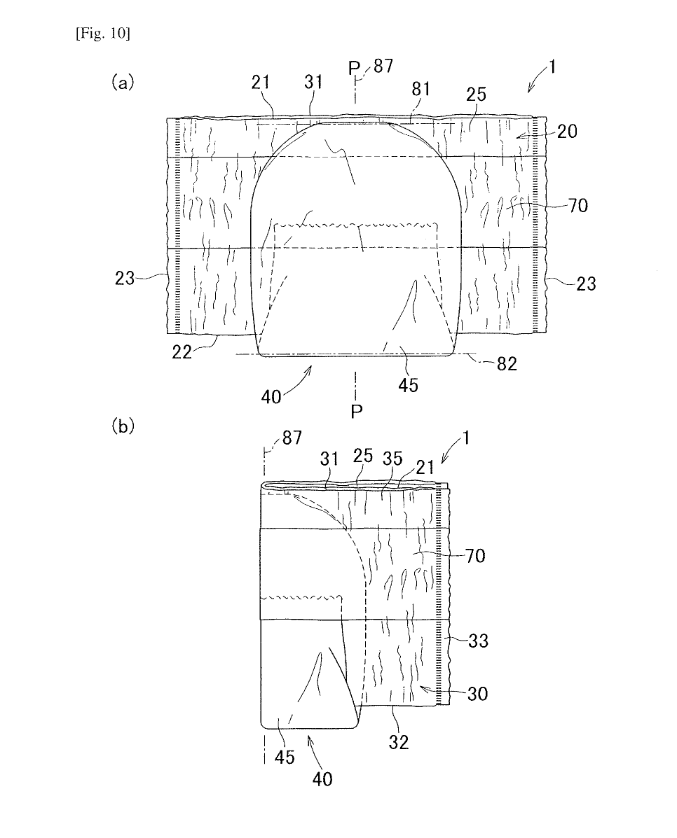

FIGS. 10(a) and 10(b) include diagrams exemplarily illustrating another procedure of folding the diaper 1 wherein FIG. 10(a) indicates locations of respective fold lines and FIG. 10(b) illustrates the diaper 1 having been folded along the fold lines. The diaper 1 is folded along the first fold line 81 and the second fold line 82, respectively, and further folded along a third fold line 87 extending in the longitudinal direction Y. The third fold line 87 corresponds to the imaginary longitudinal center line P-P or in a vicinity thereof. In the diaper 1 having been folded in this manner, the outer sheet 25 of the front waist panel 20 faces itself and the outer sheet 35 of the rear waist panel 30 is exposed. The diaper 1 may be folded in this manner to make the diaper 1 smaller and a plurality of these diapers may be stacked one on another to make the packaged assembly more compact.

By using superabsorbent polymer particles as the core material 56 of the bodily fluid absorbent member 50, the thickness dimension of the diaper 1 can be reduced and the thickness dimension of the crotch midsection 83 can be reduced. In this way, even if such diaper 1 is folded two or more times, the dimension in the thickness direction thereof is unlikely to be significantly increased. In other words, it is possible to prevent the packaged assembly from becoming significantly bulky even if the respective diapers 1 are further folded along the third fold line 87.

While FIG. 10 illustrates the diaper 1 being folded along the third fold line 87 so that the outer sheet 25 of the front waist panel 20 may face itself, it is also possible to fold the diaper 1 so that the outer sheet 35 of the rear waist panel 30 may face itself. In this case, the outer sheet 25 of the front waist panel 20 and the outer sheet 45 of the intermediate panel 40 will be exposed. When these diapers 1 respectively folded along the third fold line 87 are stacked one on another, the diapers 1 may be stacked one on another so that the outer ends 21, 31 of the adjacent diapers 1 may overlap each other or not overlap each other.

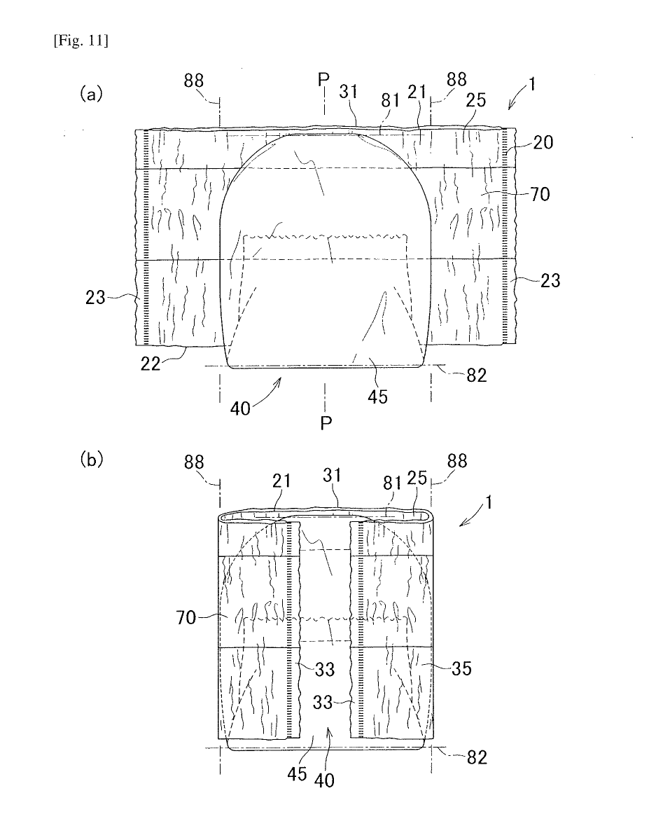

FIGS. 11(a) and 11(b) include diagrams exemplarily illustrating still another procedure of folding the diaper 1 wherein FIG. 11(a) indicates locations of respective fold lines and FIG. 11(b) illustrates the diaper 1 having been folded along the fold lines. The diaper 1 is folded along the first fold line 81 and the second fold line 82 extending in the transverse direction X, respectively, and is further folded along a pair of fourth fold lines 88 extending in the longitudinal direction Y. The fourth fold lines 88 are spaced apart from each other in the transverse direction X and lie outside the intermediate panel 40 as viewed in the transverse direction X. In the diaper 1 having the front and rear waist panels 20, 30 folded along the fourth fold lines 88, the outer sheet 25 of the front waist panel 20 faces the outer sheet 45 of the intermediate panel 40. By folding the diaper 1 in this manner, the diaper 1 can be made further small and a plurality of these diapers 1 may be stacked one on another to make the packaged assembly more compact.

Positions of the third fold line 87 and the fourth fold lines 88 both extending in the longitudinal direction Y may be appropriately selected and not limited to the above-described positions. Furthermore, it is also possible to fold the diaper 1, in addition to the fold lines extending in the longitudinal direction Y, along still another fold line extending in the transverse direction X.

Disclosures described hereinabove may be summarized at least as follows.

The first aspect of this disclosure relates to a disposable wearing article having a longitudinal direction and a transverse direction, including:

a chassis comprising a skin-facing side, a non-skin-facing side opposite to the skin-facing side, front and rear waist regions, a crotch region extending between these front and rear waist regions, a waist-opening, a pair of leg-openings, an imaginary longitudinal center line bisecting a dimension of the chassis in the transverse direction, and an imaginary transverse center line bisecting a dimension of the chassis in the longitudinal direction;

a bodily fluid absorbent member lying at least in the crotch region; and

a first fold line corresponding to or in a vicinity of the imaginary transverse center line, wherein the wearing article is folded along the first fold line so that the skin-facing side of the front waist region faces the skin-facing side of the rear waist region and the skin-facing side of the crotch region faces itself, wherein:

a crotch midsection included within the crotch region and lying on the imaginary transverse center line has a thickness dimension smaller than a thickness dimension of absorbent-free regions which are defined in the front and rear waist regions and in which the bodily fluid absorbent member is not present.

The first aspect may include at least the following embodiments.

(1) The chassis has a second fold line extending across the crotch region in the transverse direction, and the crotch region is folded along this second fold line onto one of the front and rear waist regions so that the non-skin-facing side of said one of the front and rear waist regions faces the non-skin-facing side of the crotch region.

(2) The chassis has also a third fold line extending in the longitudinal direction and corresponding to or in a vicinity of the imaginary longitudinal center line, and the front and rear waist regions and the crotch region are folded along the third fold line.

(3) The chassis additionally includes a pair of fourth fold lines extending in the longitudinal direction and spaced apart from each other in the transverse direction, and the front and rear waist regions are folded along these fourth fold lines.

(4) The chassis includes front and rear waist panels respectively defining the front and rear waist regions and adapted to be elastically stretchable and contractible in the transverse direction, and an intermediate panel defining the crotch region and portions of the front and rear waist regions, and wherein front and rear ends of the intermediate panel are joined to the front and rear waist panels, respectively, and the second fold line lies in the intermediate panel.

(5) The front and rear waist panels respectively include an inner sheet lying on the skin-facing side, an outer sheet lying on the non-skin-facing side and a plurality of waist elastics disposed between the respective inner and outer sheets so as to extend in the transverse direction and attached under tension to these inner and outer sheets so that a difference between the maximum value and the minimum value of a stretch ratio of the waist elastics is in a range of 0 to 10%.

(6) The bodily fluid absorbent member includes a liquid-permeable inner sheet lying on the skin-facing side, an outer sheet lying on the non-skin-facing side and absorbent core material disposed between these sheets.

(7) The bodily fluid absorbent member further includes stabilizing regions adapted to restrict movement of the core material.

(8) The chassis further includes leg elastics extending in the crotch region in the longitudinal direction and attached thereto under tension, and two sets of these leg elastics are arranged in vicinities of the opposite side edges of the crotch regions, respectively, so that the elastic members in the respective sets are spaced apart from each other without being continuous with each other.

The second aspect of this disclosure relates to a packaged assembly, including:

a package sheet; and

a plurality of disposable wearing articles stacked one on another in the thickness direction of the respective disposable wearing articles and packaged with the package sheet, wherein

each of these disposable wearing articles has a longitudinal direction and a transverse direction and includes:

a chassis comprising a skin-facing side, a non-skin-facing side opposite to the skin-facing side, front and rear waist regions, a crotch region extending between these front and rear waist regions, a waist-opening, a pair of leg-openings, an imaginary longitudinal center line bisecting a dimension of the chassis in the transverse direction, and an imaginary transverse center line bisecting a dimension of the chassis in the longitudinal direction;

a bodily fluid absorbent member lying at least in the crotch region; and

a first fold line corresponding to or in a vicinity of the imaginary transverse center line, wherein

a crotch midsection included within the crotch region and lying on the imaginary transverse center line has a thickness dimension smaller than a thickness dimension of absorbent-free regions which are defined in the front and rear waist regions and in which the bodily fluid absorbent member is not present, and

each of the wearing articles is folded along the first fold line so that the skin-facing side of the front waist region faces the skin-facing side of the rear waist region and the skin-facing side of the crotch region faces itself.

The second aspect of this disclosure may include at least the following embodiments.

(1) In each of the disposable wearing articles, the chassis has a second fold line extending across the crotch region in the transverse direction, and the crotch region is folded along the second fold line onto one of the front and rear waist regions so that the non-skin-facing side of said one of the front and rear waist regions faces the non-skin-facing side of the crotch region.

(2) Each of the disposable wearing articles has opposite first and second surfaces, the folded crotch region is exposed more on the first surface than on the second surface, and

the adjacent wearing articles are stacked on each other so that the respective first surfaces face each other.

(3) In each of the disposable wearing articles, the chassis has outer ends cooperating with each other to define the waist-opening and the wearing article has an outer shape defined by the outer ends, the second fold line and the opposite side edges of the chassis; and

the adjacent wearing articles are stacked on each other so that the outer ends of the one of the adjacent wearing articles faces the second fold line of the other of the adjacent wearing articles.

(4) The package sheet has an upper surface, a bottom surface and a side surface extending between these upper and bottom surfaces; and the disposable wearing articles are stacked one on another between the upper surface and the bottom surface, and the upper surface is formed with an indicator line along which the package sheet is tearable.

According to one or more aspects and/or embodiments of this invention, a thickness dimension of the crotch midsection on the imaginary longitudinal center line in the crotch region is adjusted so as to be smaller than that of the absorbent-free regions defined in the front and rear waist regions and not occupied by the bodily fluid absorbent member. In this way, the crotch region folded on itself should not become significantly thicker than the other regions.

The constituent members of the diaper 1 are not limited to those described in the specification but the other various types of material widely used in the relevant technical field may be used without limitation unless otherwise state. The terms “first”, “second”, “third” and “fourth” used in the specification and claims of this application are used merely to distinguish similar elements, similar positions or similar means.

This application claims the benefit of Japanese Applications Nos. 2011-146655 and 2011-261124 the entire disclosure of which is incorporated by reference herein.

1. A disposable wearing article having a longitudinal direction and a transverse direction, including:
a chassis comprising a skin-facing side, a non-skin-facing side opposite to the skin-facing side, front and rear waist regions, a crotch region extending between these front and rear waist regions, a waist-opening, a pair of leg-openings, an imaginary longitudinal center line bisecting a dimension of the chassis in the transverse direction, and an imaginary transverse center line bisecting a dimension of the chassis in the longitudinal direction; a bodily fluid absorbent member lying at least in the crotch region; and a first fold line corresponding to or in a vicinity of the imaginary transverse center line, wherein the wearing article is folded along the first fold line so that the skin-facing side of the front waist region faces the skin-facing side of the rear waist region and the skin-facing side of the crotch region faces itself, wherein:
a crotch midsection included within the crotch region and lying on the imaginary transverse center line has a thickness dimension smaller than a thickness dimension of absorbent-free regions which are defined in the front and rear waist regions and in which the bodily fluid absorbent member is not present.

2. The disposable wearing article defined by claim 1, wherein the chassis has a second fold line extending across the crotch region in the transverse direction, and the crotch region is folded along this second fold line onto one of the front and rear waist regions so that the nonskin-facing side of said one of the front and rear waist regions faces the non-skin-facing side of the crotch region.

3. The disposable wearing article defined by claim 2, wherein the chassis has also a third fold line extending in the longitudinal direction and corresponding to or in a vicinity of the imaginary longitudinal center line, and the front and rear waist regions and the crotch region are folded along the third fold line.

4. The disposable wearing article defined by claim 2, wherein the chassis additionally includes a pair of fourth fold lines extending in the longitudinal direction and spaced apart from each other in the transverse direction, and the front and rear waist regions are folded along these fourth fold lines.

5. The disposable wearing article defined by, claim 1, wherein the chassis includes front and rear waist panels respectively defining the front and rear waist regions and adapted to be elastically stretchable and contractible in the transverse direction, and an intermediate panel defining the crotch region and portions of the front and rear waist regions, and wherein front and rear ends of the intermediate panel are joined to the front and rear waist panels, respectively, and the second fold line lies in the intermediate panel.

6. The disposable wearing article defined by claim 1, wherein the front and rear waist panels respectively include an inner sheet lying on the skin-facing side, an outer sheet lying on the nonskin-facing side and a plurality of waist elastics disposed between the respective inner and outer sheets so as to extend in the transverse direction and attached under tension to these inner and outer sheets so that a difference between the maximum value and the minimum value of a stretch ratio of the waist elastics is in a range of 0 to 10%.

7. The disposable wearing article defined by claim 1, wherein the bodily fluid absorbent member includes a liquid-permeable inner sheet lying on the skin-facing side, an outer sheet lying on the non-skin-facing side and absorbent core material disposed between these sheets.

8. The disposable wearing article defined by claim 7, wherein the bodily fluid absorbent member further includes stabilizing regions adapted to restrict movement of the core material.

9. The disposable wearing article defined by claim 1, wherein the chassis further includes leg elastics extending in the crotch region in the longitudinal direction and attached thereto under tension, and two sets of these leg elastics are arranged in vicinities of the opposite side edges of the crotch regions, respectively, so that the elastic members in the respective sets are spaced apart from each other without being continuous with each other.

10. A packaged assembly, comprising:
a package sheet; and a plurality of disposable wearing articles stacked one on another in the thickness direction of the respective disposable wearing articles and packaged with the package sheet, wherein each of these disposable wearing articles has a longitudinal direction and a transverse direction and includes: a chassis comprising a skin-facing side, a non-skin-facing side opposite to the skin-facing side, front and rear waist regions, a crotch region extending between these front and rear waist regions, a waist-opening, a pair of leg-openings, an imaginary longitudinal center line bisecting a dimension of the chassis in the transverse direction, and an imaginary transverse center line bisecting a dimension of the chassis in the longitudinal direction;
a bodily fluid absorbent member lying at least in the crotch region; and
a first fold line corresponding to or in a vicinity of the imaginary transverse center line, wherein a crotch midsection included within the crotch region and lying on the imaginary transverse center line has a thickness dimension smaller than a thickness dimension of absorbent-free regions which are defined in the front and rear waist regions and in which the bodily fluid absorbent member is not present, and each of the wearing articles is folded along the first fold line so that the skin-facing side of the front waist region faces the skin-facing side of the rear waist region and the skin-facing side of the crotch region faces itself.

11. The packaged assembly defined by claim 10, wherein, in each of the disposable wearing articles, the chassis has a second fold line extending across the crotch region in the transverse direction, and the crotch region is folded along the second fold line onto one of the front and rear waist regions so that the non-skin-facing side of said one of the front and rear waist regions faces the non-skin-facing side of the crotch region.

12. The packaged assembly defined by claim 11, wherein each of the disposable wearing articles has opposite first and second surfaces, the folded crotch region is exposed more on the first surface than on the second surface, and the adjacent wearing articles are stacked on each other so that the respective first surfaces face each other.

13. The packaged assembly defined by claim 11, wherein:
in each of the disposable wearing articles, the chassis has outer ends cooperating with each other to define the waist-opening and the wearing article has an outer shape defined by the outer ends, the second fold line and the opposite side edges of the chassis; and the adjacent wearing articles are stacked on each other so that the outer ends of the one of the adjacent wearing articles faces the second fold line of the other of the adjacent wearing articles.

14. The packaged assembly defined by claim 10 wherein: the package sheet has an upper surface, a bottom surface and a side surface extending between these upper and bottom surfaces; and the wearing articles are stacked one on another between the upper surface and the bottom surface, and the upper surface is formed with an indicator line along which the package sheet is tearable.
